(19) United States
(12) Patent Application Publication
(10) Pub. No.: US 2020/0079853 A1
(43) Pub. Date: Mar. 12, 2020

(54) T REGULATORY CELLS AND USES THEREOF

(71) Applicant: Junten Bio Co., Ltd., Tokyo (JP)

(72) Inventors: Satoru Todo, Kurume City (JP); Kenichiro Yamashita, Sapporo City (JP); Hisashi Bashuda, Tokyo (JP); Ko Okumura, Chiba City (JP)

(21) Appl. No.: 16/680,405

(22) Filed: Nov. 11, 2019

Related U.S. Application Data
(63) Continuation of application No. 14/783,258, filed on Oct. 8, 2015.
(60) Provisional application No. 61/824,590, filed on May 17, 2013.

(51) Int. Cl. C07K 16/28; A61K 45/06; A61K 35/17; C12N 5/0783; A61K 39/00; A61K 31/675; A61K 31/198; A61K 38/13; A61K 31/365; A61K 31/56; A61K 31/453

(52) U.S. Cl. CPC C07K 16/2827; A61K 2039/507; A61K 35/17; C12N 5/0637; A61K 39/001; A61K 31/675; A61K 31/198; A61K 38/13; A61K 31/365; A61K 31/56; A61K 31/453; C07K 2317/76; C07K 2317/31; C12N 2501/51; A61K 2039/5158; A61K 45/06

(57) ABSTRACT

The present application relates to generation of regulatory T cells. The present application also relates to uses of the regulatory T cells in treating subjects undergoing organ transplantation. The present application also relates to uses of the regulatory T cells in treating subjects undergoing tissue grafts. Regulatory T cells may be administered to a subject along with one or more antibodies.

### CROSS-REFERENCE TO RELATED APPLICATIONS

This application is a continuation application of U.S. patent application Ser. No. 14/783,258, filed Oct. 8, 2015, which is a National Stage Entry of PCT Application No. PCT/US2014/037195, filed May 7, 2014, which claims the benefit of U.S. Provisional Application No. 61/824,590, filed May 17, 2013, which is incorporated herein by reference in its entirety.

### BACKGROUND OF THE INVENTION

Liver transplantation has been widely used as an ultimate treatment for patients with end-stage liver failure. There are more than 20,000 cases overseas and more than 500 cases in Japan every year.

Transplantation is one of the main treatments of choice for end stage kidney, heart, liver and pancreas organ failure and despite considerable advances in the management of transplant rejection in recent years the vast majority of transplants are eventually rejected. Current immunosuppressive regimens, which depend on continual drug therapy, predispose transplant patients to increased susceptibility to infections and cancer because even the drugs are unable to inhibit those responses specifically directed toward the transplant.

### SUMMARY OF THE INVENTION

Provided herein is a method of treating a condition in a subject mediated by an immune response which comprises administering to said subject a composition comprising antibodies, or antigen-binding fragments thereof, that specifically bind to CD80 and CD86 to generate a population of regulatory T-lymphocytes.

In one embodiment, the composition comprises antibodies that specifically bind to CD80 and antibodies that specifically bind to CD86.

In another embodiment, the antibodies bind to one or more epitopes on CD80 and one or more epitopes on CD86.

In yet another embodiment, the antibodies block and/or neutralize CD80 and CD86.

Also provided herein is an ex vivo method for generating a population of regulatory T lymphocytes comprising culturing T cells with a composition comprising antibodies that specifically bind to CD80 and CD86 in the presence of cells that present either alloantigen or a non-cellular protein antigen.

In one embodiment, the non-cellular protein antigen is human gamma globulin, equine gamma globulin or ovalbumin.

In another embodiment, the T cells are taken from a recipient animal and the cells that present alloantigen are either cells taken from a donor animal or cells pulsed with antigen taken from a donor animal.

Also provided herein is a cell culture prepared by the method described above, comprising culturing cells obtained by the ex vivo method in medium. In one embodiment, the antibodies that specifically bind to CD80 and CD86 are removed from said medium. The antibodies may be removed from the culture medium by, for example, by washing the cells and reconstituting them in medium. The cells obtained from these methods may be further administered to a recipient subject in need thereof.

Also provided herein is a method of suppressing rejection of an organ or tissue transplant in a recipient subject comprising the following steps: (a) obtaining a sample of T cells from the recipient subject; (b) obtaining a sample of alloantigen from a donor subject, said donor subject being the source of the organ or tissue being transplanted; (c) exposing said sample of T cells to said sample of alloantigen in the presence of a composition comprising antibodies that specifically bind to CD80 and CD86 to generate a composition comprising a population of regulatory T lymphocytes; and (d) administering to the recipient subject a composition comprising said population of regulatory T-lymphocytes. Step (c) may further comprise removing the antibodies from said composition prior to step (d).

In one embodiment, from about 1×10⁹ to about 1×10¹⁵ cells may be administered to said recipient subject. The population of regulatory T-lymphocytes is administered to the recipient subject prior to, concurrently with, or after, transplant of an organ or tissue. In one embodiment, the subject is a human.

In another embodiment, the method may further comprise administering to the recipient subject one or more immunosuppressive drugs. Non-limiting examples of immunosuppressive drugs include, for example, a calcineurin inhibitor (e.g., tacrolimus (FK-506), cyclosporine A (CsA), etc.), adriamycin, azathiopurine (AZ) , busulfan, cyclophosphamide, deoxyspergualin (DSG); FTY720 (also called Fingolimod, chemical name: 2-amino-2-[2-(4-octylphenyl)ethyl]-1,3-propanediol hydrochloride), fludarabine, 5-fluorouracil, leflunomide (LEF); methotrexate, mizoribine (MZ), mycophenolate mofetil (MMF), a nonsteroidal anti-inflammatory, sirolimus (rapamycin), an adrenocortical steroid (e.g., prednisolone and methylprednisolone), agents that block CTLA-4 and/or CD28, an antibody (e.g., muromonab-CD3, alemtuzumab, basiliximab, daclizumab, rituximab, anti-thymocyte globulin, etc), and combinations thereof. The one or more immunosuppressive drugs may be administered to the recipient subject prior to, concurrently with, or after, transplant of an organ or tissue.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the embodiments are set forth with particularity in the appended claims. A better understanding of the features and advantages of the present embodiments will be obtained by reference to the following detailed description that sets forth illustrative embodiments, in which the principles of the embodiments are utilized, and the accompanying drawings of which:

FIGS. 1A-D show the results of flow cytometry regarding surface antigen analysis before and after a typical culture. Regulatory T cells markers appeared in human peripheral cells post-culture with the irradiated second party human ones (stimulator) in the presence of anti-CD80 and anti-CD86 antibodies. Flow cytometry plots showed antigen reactive cells stained for CD25 and FoxP3 (top; FIGS. 1A and 1B) and for CD25 and CTLA-4 (bottom FIGS. 1C and 1D) on day 0 (left panels FIGS. 1A and 1C) and 14 (right panels FIGS. 1B and 1D) after culture.

FIGS. 2A-B illustrate the suppressive effect by cultured cells with CD80 and CD86 in a mixed lymphocyte reaction (MLR). In one-way MLR (in adding previously cultured second party cells with CD80 and CD86), fresh human peripheral cells are suppressed proliferative response to the irradiated second party cells (upper graph; FIG. 2A) but showed good response to the irradiated third party cells (lower graph; FIG. 2B), indicating that suppressive effect is antigen specific. Those experiments are done using peripheral white cells of two healthy persons. FIG. 2A shows the MLR result using the donor's antigen (radiated lymphocytes) used in culture of regulatory T cells, and FIG. 2B shows the MLR result of antigen from a third party's donor (radiated lymphocytes). Columns 1 to 3 on each graph show the cell proliferation when the recipient's lymphocytes, radiated lymphocytes and 2-week cultured lymphocytes are individually cultured. Column 4 is the cell proliferation of recipient's lymphocyte upon addition of co-culturing and simulating with donor's antigen (radiated lymphocytes) (control). The results of adding 1/1, ½ and ¼ amount of cultured cells in this system are shown in columns 5 to 7, but by adding the cultured cells, the proliferation of lymphocytes is strongly inhibited, and addition of cell count reveals a strong immunosuppressive effect even in ¼ (0.25×105).

FIG. 3 provides ELISA results for the various studies which demonstrates that the anti-CD80 and anti-CD86 antibodies are removed from the culture medium after serial washing.

FIGS. 4A-D show the results of flow cytometry regarding surface antigen analysis before and after a typical culture. Regulatory T cell markers are induced in recipient T cells by co-cultured with donor cells (antigen stimulator) in the presence of CD80 and CD86. By induction methods described in FIG. 2, recipient peripheral cells were cultured with irradiated donor cells in the presence of antibodies to CD80/86. Their phenotypes were also CD25+Foxp3+(upper panels; FIGS. 4A and 4B) and CD25+CTLA-4+(lower panels; FIGS. 4C and 4D) on days 0 (left panels; FIGS. 4A and 4C) and 14 (right panels; FIGS. 4B and 4D) of culture.

FIGS. 5A-B provide an illustration of a treatment regiment and CsA levels post-transplantation. FIG. 5A provides a schematic diagram of the regimen (group A). CsA (8 mg/kg/d) was administered intramuscularly on the days indicated by asterisks. CP (30 mg/kg) was administered intramuscularly at PODs 6, 7, and 8. During the operation, the spleen was removed from both donor and recipient. Splenic T cells from the recipient were co-cultured with irradiated donor splenocytes for 13 days in the presence of anti-CD80/CD86 mAbs and injected into the recipient. No further immunosuppression was given thereafter. FIG. 5B provides a graph illustrating CsA whole blood levels (ng/ml) in recipients (group A). CsA (8 mg/kg) was injected intramuscularly daily from the day of operation to 7 days after transplantation and thereafter on days 9, 11, and 13. Solid lines, animals surviving more than 1 year (n=3); dotted lines, animals dying within 1 year (n=3).

FIGS. 6A-B provide an illustration of the protocol for Case 2 as described in the examples. Briefly, 1.5×10⁹ regulatory T cells are infused at week 2 after surgery, and the immunosuppressive agent (cyclosporin (CYA): 300 mg/day, mycophenolate mofeteil (MMF): 2000 mg/day, methylprednisolone (MP): 500 mg/day) are reduced gradually (FIG. 6A). Following treatment, the doses of day 225 are CYA 50 mg/day and MMF 500 mg/day, and MP is completely discontinued (FIG. 6B).

FIGS. 7A-D provide flow cytometry of induced regulatory T cells of the protocol of case 2 illustrated in FIG. 6. This figure shows the CD25+Foxp3⁺ cells (upper panels; FIGS. 7A and 7B) and CD25+ CTLA4⁺ cells (lower panels; FIGS. 7C and 7D) in a cell population that has been sorted for CD4⁺ cells on days 0 (left panels; FIGS. 7A and 7C) and after 2 weeks of culture (right panels; FIGS. 7B and 7D).

FIGS. 8A-B provides the results of the MLR inhibitory effect of case 5. The regulatory cells, which are tested in vitro (FIG. 8A) and in kidney transplant patients (case 5; FIG. 8B), inhibit the donor's antigen-specific lymphocyte proliferation.

### DETAILED DESCRIPTION OF THE INVENTION

Transplant rejection occurs when transplanted tissue is rejected by a recipient's immune system, which destroys the transplanted tissue. Transplant rejection can be lessened by determining the molecular similitude between donor and recipient and by use of immunosuppressant drugs after transplant. The present inventors have identified a new subset of T regulatory cells that are useful for suppressing rejection of organ transplants.

### Problems of Conventional Immunosuppressive Therapies in Liver Transplantation

Liver transplantation has been widely used as an ultimate treatment for patients with end-stage liver failure. This development of liver transplantation depends on the advancement in surgical techniques, organ preservation, and pre- and post-operative management etc., but above all, the improvement in immunosuppressive agent has greatly contributed to it. The 1-year and 5-year survival rates have been dramatically increased by 35% and 20%, 70 and 60%, and 80% and 70% respectively due to azathioprine (1960-70s), cyclosporine (1980's) and tacrolimus (since 1990's) that have been used (Refs. 1, 2). The world's first clinical liver transplantation was performed in 1963; to date, more than 300,000 cases have been performed with more than 20,000 cases overseas and more than 500 cases in Japan every year. There are important, unresolved issues that impact both medicine and the medical economy because these patients must take lifelong immunosuppressive agents to control the rejection, and are always exposed to the risk of drug-induced side effects such as infectious diseases and carcinogenesis. To eliminate these problems, a so-called induction of immune tolerance is required so that the graft functions properly even if the immunosuppressive agent is discontinued.

CD4+ T-helper (T_{H}) lymphocytes are cells that, in healthy individuals, play an essential role in immune responses that protect subjects from pathogenic organisms such as, for example, bacteria and viruses. However, when a subject receives a transplanted organ, these cells mainly cause rejection of organ transplants. It was previously determined that rejection of the organ transplant could be attenuated by administration of immunosuppressive agents, such as anti-CD4 antibodies which target CD4+ T cells. Recently, it was shown that antibody therapy can, in some instances, lead to the generation of sub-populations of T cells that can control or regulate adverse rejection responses. Regulatory cells may be generated in such instances because the presence of the anti-CD4 antibody prevents full T cell activation, and the cells default to a regulatory or suppressive phenotype.

### Immune Tolerance and Regulatory T Cells

From the early 1970's, inhibitory (suppressor) T cells have been found in recipients with a state of immune tolerance in the models of autoimmune disease and organ transplantation using small animals, and this lymphocyte can transfer the immune tolerance (infectious tolerance) by adoptive transfer to a naïve host.

These findings have advanced the study of suppressor T cells. Okumura (Ref 3), a co-investigator is the first person in the field of transplantation immunology who announced this suppressor T cell for the first time in the world in the early 1970's. Studies on suppressor T cells have declined once because it was difficult to establish that cytological identification method, but recently, phenotype/markers such as CD4⁺ CD25⁺ and Foxp3⁺ have been discovered, regulatory T cell: Treg has attracted attention again (Ref 4), and the efficacy using the same cells focusing on in vitro and small animal transplantation model has been reported in many studies.

### Ex vivo Induction and Proliferation of Regulatory T Cells

Graft rejection of graft in allogeneic organ transplantation is mainly caused by cell-mediated immunity of the recipient. As this cell-mediated immunity is a donor antigen specific reaction, the donor antigen presented by antigen presenting cells such as dendritic cells recognizes the helper CD4 T cells and the effector CD8 T cells are activated, and rejection is ultimately caused. Co-stimulation is required in activation of helper T cells was known for the first time in the early 1990s, but the group of Okumura et al. have found that the co-stimulation is transmitted by binding of CD28 on T cells to antigen-presenting cells on CD80/CD86 on antigen-presenting cells, and the recipient T cells do not cause an immune response against the donor antigen-presenting cells by adding anti-CD80 antibody and anti-CD86 antibody in the cell culture, as a result, leading to a state of donor's antigen-specific anergy. In a recent study, the anergy T cell has been found to act as the donor's antigen-specific regulatory T cell (Treg). In addition, the co-investigators Okumura, Bashuta, Seino et al. have succeeded in inducing the antigen specific Treg-like cells ex vivo by adding anti-CD80 and anti-CD86 antibodies in lymphocyte culture medium, and have confirmed the long-term graft survival in mouse heart transplantation model by infusing the harvested cells (Ref 6). Moreover, in a study trying the same protocol for preclinical studies using the monkey kidney transplantation model, by infusion of Treg-like cells harvested from co-culturing and inducing peripheral blood mononuclear cells (PBMC) under the donor's splenocytes and anti-CD80/CD86 antibodies, into the recipient before and after 2 weeks after transplantation, the early withdrawal of immunosuppressive agent, cyclosporine has been enabled, and the transplanted kidney has achieved long-term (>600 days) engraftment and the donor's antigen specific immune tolerance has been successfully induced even in a immunosuppression free state (Ref. 7).

Antibodies, Generation of T Regulatory Cells, Cell Cultures and Uses Thereof

Provided herein is a method of treating a condition in a subject mediated by an immune response which comprises administering to said subject a composition comprising antibodies, or antigen-binding fragments thereof, that specifically bind to CD80 and CD86 to generate a population of regulatory T-lymphocytes.

In one embodiment, the composition comprises antibodies that specifically bind to CD80 and antibodies that specifically bind to CD86.

In another embodiment, the antibodies bind to one or more epitopes on CD80 and one or more epitopes on CD86.

In yet another embodiment, the antibodies block and/or neutralize CD80 and CD86.

In yet another embodiment, an antigen-binding fragment may be, for example, a Fab fragment, a Fab′ fragment, a F(ab′)₂ fragment, an Fv fragment, an scFv fragment, a single chain binding polypeptide, a Fd fragment, a variable heavy chain, a variable light chain, a dAb fragment, an AVIMER, a diabody, or a heavy chain dimer. A heavy chain dimer may be, for example, a camelid or a shark heavy chain construct.

Any antibody that specifically binds to CD80 or CD86 may be used in the compositions described herein such as those found, for example, in the examples below. Commercially available antibodies and hybridomas may be obtained, for example, from ATCC; variable heavy and light chain sequences may be found in public databases such as, for example, NCBI PubMed; and companies such as Bay Bioscience Co., Ltd., Thermo Scientific Pierce Antibodies, LifeSpan Biosciences, Inc., and BD Biosciences, also commercially produce anti-CD80 and anti-CD86 antibodies.

As used herein, the term “antibody” refers to an immunoglobulin (Ig) whether natural or partly or wholly synthetically produced. The term also covers any polypeptide or protein having a binding domain which is, or is homologous to, an antigen-binding domain. The term further includes “antigen-binding fragments” and other interchangeable terms for similar binding fragments such as described below.

Native antibodies and native immunoglobulins are usually heterotetrameric glycoproteins of about 150,000 Daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}” or “VH”) followed by a number of constant domains (“C_{H}” or “CH”). Each light chain has a variable domain at one end (“V_{L}” or “VL”) and a constant domain (“C_{L}” or “CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The terms “synthetic polynucleotide,” “synthetic gene” or “synthetic polypeptide,” as used herein, mean that the corresponding polynucleotide sequence or portion thereof, or amino acid sequence or portion thereof, is derived, from a sequence that has been designed, or synthesized de novo, or modified, compared to an equivalent naturally-occurring sequence. Synthetic polynucleotides (antibodies or antigen binding fragments) or synthetic genes can be prepared by methods known in the art, including but not limited to, the chemical synthesis of nucleic acid or amino acid sequences. Synthetic genes are typically different from naturally-occurring genes, either at the amino acid, or polynucleotide level, (or both) and are typically located within the context of synthetic expression control sequences. Synthetic gene polynucleotide sequences, may not necessarily encode proteins with different amino acids, compared to the natural gene; for example, they can also encompass synthetic polynucleotide sequences that incorporate different codons but which encode the same amino acid (i.e., the nucleotide changes represent silent mutations at the amino acid level).

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3 and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669).

The terms “hypervariable region” and “CDR” when used herein, refer to the amino acid residues of an antibody which are responsible for antigen-binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2) and 89-97 (CDRL3), and in the heavy chain variable domain the CDRs typically correspond to approximately residues 31-35 (CDRH1), 50-65 (CDRH2) and 95-102 (CDRH3) according to Kabat et al., Sequences of Proteins of Immunological Interest, 5th Ed. Public Health Service, National Institutes of Health, Bethesda, Md. (1991)). It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2) and 91-96 (CDRL3), and in the heavy chain variable domain, the CDRs typically correspond to approximately residues 26-32 (CDRH1), 53-55 (CDRH2) and 96-101 (CDRH3) according to Chothia and Lesk, J. Mol. Biol., 196: 901-917 (1987)).

As used herein, “framework region” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al., Sequences of Proteins of Immunological Interest, 5th Ed. Public Health Service, National Institutes of Health, Bethesda, Md. (1991)). As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, J. Mol. Biol., 196: 901-917 (1987)).

Constant domains (Fc) of antibodies are not involved directly in binding an antibody to an antigen but, rather, exhibit various effector functions, such as participation of the antibody in antibody-dependent cellular toxicity via interactions with, for example, Fc receptors (FcR). Fc domains can also increase bioavailability of an antibody in circulation following administration to a patient. Substitution of a murine Fc domain for a human Fc domain can also reduce side HAMA reactions.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgAl, and IgA2. The heavy-chain constant domains (Fc) that correspond to the different classes of immunoglobulins are called α, δ, ε, γ, and μ, respectively. The subunit structures and three-dimensional configurations of different classes of immunoglobulins are well known.

The “light chains” of antibodies from any vertebrate species can be assigned to one of two clearly distinct types, called kappa or (“κ”) and lambda or (λ), based on the amino acid sequences of their constant domains.

The terms “antigen-binding portion of an antibody,” “antigen-binding fragment,” “antigen-binding domain,” “antibody fragment” or a “functional fragment of an antibody” are used interchangeably herein to refer to one or more fragments of an antibody that retain the ability to specifically bind to an antigen. Non-limiting examples of antibody fragments included within such terms include, but are not limited to, (i) a Fab fragment, a monovalent fragment consisting of the V_{L}, V_{H}, C_{L} and C_{H1} domains; (ii) a F(ab′)₂ fragment, a bivalent fragment containing two Fab fragments linked by a disulfide bridge at the hinge region; (iii) a Fd fragment consisting of the V_{H} and C_{H1} domains; (iv) a Fv fragment containing the V_{L} and V_{H} domains of a single arm of an antibody, (v) a dAb fragment (Ward et al., (1989) Nature 341:544 546), which containing a V_{H} domain; and (vi) an isolated CDR. Additionally included in this definition are “one-half” antibodies comprising a single heavy chain and a single light chain. Other forms of single chain antibodies, such as diabodies are also encompassed herein.

“F(ab′)₂” and “Fab′” moieties can be produced by treating an Ig with a protease such as pepsin and papain, and include antibody fragments generated by digesting immunoglobulin near the disulfide bonds existing between the hinge regions in each of the two heavy chains. For example, papain cleaves IgG upstream of the disulfide bonds existing between the hinge regions in each of the two heavy chains to generate two homologous antibody fragments in which an light chain composed of V_{L} and C_{L} (light chain constant region), and a heavy chain fragment composed of V_{H} and C_{Hγ1} (λ1) region in the constant region of the heavy chain) are connected at their C terminal regions through a disulfide bond. Each of these two homologous antibody fragments is called Fab′. Pepsin also cleaves IgG downstream of the disulfide bonds existing between the hinge regions in each of the two heavy chains to generate an antibody fragment slightly larger than the fragment in which the two above-mentioned Fab′ are connected at the hinge region. This antibody fragment is called F(ab′)₂.

The Fab fragment also contains the constant domain of the light chain and the first constant domain (C_{H}1) of the heavy chain. Fab′ fragments differ from Fab fragments by the addition of a few residues at the carboxyl terminus of the heavy chain C_{H}1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” refers to an antibody fragment which contains a complete antigen-recognition and antigen-binding site. This region consists of a dimer of one heavy chain and one light chain variable domain in tight, non-covalent or covalent association (disulfide linked Fv's have been described in the art, Reiter et al. (1996) Nature Biotechnology 14:1239-1245). It is in this configuration that the three CDRs of each variable domain interact to define an antigen-binding site on the surface of the V_{H}-V_{L} dimer. Collectively, a combination of one or more of the CDRs from each of the V_{H} and V_{L} chains confer antigen-binding specificity to the antibody. For example, it would be understood that, for example, the CDRH3 and CDRL3 could be sufficient to confer antigen-binding specificity to an antibody when transferred to V_{H} and V_{L} chains of a recipient antibody or antigen-binding fragment thereof and this combination of CDRs can be tested for binding, affinity, etc. using any of the techniques described herein. Even a single variable domain (or half of an Fv comprising only three CDRs specific for an antigen) has the ability to recognize and bind antigen, although likely at a lower affinity than when combined with a second variable domain. Furthermore, although the two domains of a Fv fragment (V_{L} and V_{H}), are coded for by separate genes, they can be joined using recombinant methods by a synthetic linker that enables them to be made as a single protein chain in which the V_{L} and V_{H} regions pair to form monovalent molecules (known as single chain Fv (scFv); Bird et al. (1988) Science 242:423-426; Huston et al. (1988) Proc. Natl. Acad. Sci. USA, 85:5879-5883; and Osbourn et al. (1998) Nat. Biotechnol. 16:778). Such scFvs are also intended to be encompassed within the term “antigen-binding portion” of an antibody. Any V_{H} and V_{L} sequences of specific scFv can be linked to an Fc region cDNA or genomic sequences, in order to generate expression vectors encoding complete Ig (e.g., IgG) molecules or other isotypes. V_{H} and V_{L} can also be used in the generation of Fab, Fv or other fragments of Igs using either protein chemistry or recombinant DNA technology.

“Single-chain Fv” or “sFv” antibody fragments comprise the V_{H} and V_{L} domains of an antibody, wherein these domains are present in a single polypeptide chain. In some embodiments, the Fv polypeptide further comprises a polypeptide linker between the V_{H} and V_{L} domains which enables the sFv to form the desired structure for antigen binding. For a review of sFvs see, e.g., Pluckthun in The Pharmacology of Monoclonal Antibodies, Vol. 113, Rosenburg and Moore eds. Springer-Verlag, New York, pp. 269-315 (1994).

The term “AVIMER™” refers to a class of therapeutic proteins of human origin, which are unrelated to antibodies and antibody fragments, and are composed of several modular and reusable binding domains, referred to as A-domains (also referred to as class A module, complement type repeat, or LDL-receptor class A domain). They were developed from human extracellular receptor domains by in vitro exon shuffling and phage display (Silverman et al., 2005, Nat. Biotechnol. 23:1493-1494; Silverman et al., 2006, Nat. Biotechnol. 24:220). The resulting proteins can contain multiple independent binding domains that can exhibit improved affinity (in some cases, sub-nanomolar) and specificity compared with single-epitope binding proteins. See, for example, U.S. Patent Application Publ. Nos. 2005/0221384, 2005/0164301, 2005/0053973 and 2005/0089932, 2005/0048512, and 2004/0175756, each of which is hereby incorporated by reference herein in its entirety.

Each of the known 217 human A-domains comprises ˜35 amino acids (˜4 kDa); and these domains are separated by linkers that average five amino acids in length. Native A-domains fold quickly and efficiently to a uniform, stable structure mediated primarily by calcium binding and disulfide formation. A conserved scaffold motif of only 12 amino acids is required for this common structure. The end result is a single protein chain containing multiple domains, each of which represents a separate function. Each domain of the proteins binds independently and the energetic contributions of each domain are additive. These proteins were called “AVIMERs™” from avidity multimers.

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444 6448 (1993).

Antigen-binding polypeptides also include heavy chain dimers such as, for example, antibodies from camelids and sharks. Camelid and shark antibodies comprise a homodimeric pair of two chains of V-like and C-like domains (neither has a light chain). Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called V_{HH} domains. Shark Ig-NARs comprise a homodimer of one variable domain (termed a V-NAR domain) and five C-like constant domains (C-NAR domains). In camelids, the diversity of antibody repertoire is determined by the CDRs 1, 2, and 3 in the V_{H} or V_{HH} regions. The CDR3 in the camel V_{HH} region is characterized by its relatively long length, averaging 16 amino acids (Muyldermans et al., 1994, Protein Engineering 7(9): 1129). This is in contrast to CDR3 regions of antibodies of many other species. For example, the CDR3 of mouse V_{H} has an average of 9 amino acids. Libraries of camelid-derived antibody variable regions, which maintain the in vivo diversity of the variable regions of a camelid, can be made by, for example, the methods disclosed in U.S. Patent Application Ser. No. 20050037421.

“Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin are inserted in place of the murine Fc. For details, see Jones et al., Nature 321: 522-525 (1986); Reichmann et al., Nature 332: 323-329 (1988); and Presta, Curr. Op. Struct. Biol., 2: 593-596 (1992).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which can include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, monoclonal antibodies can be made by the hybridoma method first described by Kohler et al., Nature 256:495 (1975), or can be made by recombinant DNA methods (see, e.g., U.S. Pat. No. 4,816,567). In certain embodiments, the monoclonal antibodies can be isolated from phage antibody libraries using the techniques described in Clackson et al., Nature 352:624-628 (1991) and Marks et al., J. Mol. Biol. 222:581-597 (1991), for example.

Antibodies can be isolated and purified from the culture supernatant or ascites mentioned above by saturated ammonium sulfate precipitation, euglobulin precipitation method, caproic acid method, caprylic acid method, ion exchange chromatography (DEAE or DE52), or affinity chromatography using anti-Ig column or a protein A, G or L column such as described in more detail below.

When constructing an immunoglobulin molecule, variable regions or portions thereof may be fused to, connected to, or otherwise joined to one or more constant regions or portions thereof to produce any of the antibodies described herein. This may be accomplished in a variety of ways known in the art, including but not limited to, molecular cloning techniques or direct synthesis of the nucleic acids encoding the molecules

As used herein, “immunoreactive” refers to binding agents, antibodies or fragments thereof that are specific to a sequence of amino acid residues (“binding site” or “epitope”), yet if are cross-reactive to other peptides/proteins, are not toxic at the levels at which they are formulated for administration to human use. The term “binding” refers to a direct association between two molecules, due to, for example, covalent, electrostatic, hydrophobic, and ionic and/or hydrogen-bond interactions under physiological conditions, and including interactions such as salt bridges and water bridges and any other conventional binding means. The term “preferentially binds” means that the binding agent binds to the binding site with greater affinity than it binds unrelated amino acid sequences. Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the binding agent for unrelated amino acid sequences. The terms “immunoreactive” and “preferentially binds” are used interchangeably herein.

As used herein, the term “affinity” refers to the equilibrium constant for the reversible binding of two agents and is expressed as Kd. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

“Epitope” refers to that portion of an antigen or other macromolecule capable of forming a binding interaction with the variable region binding pocket of an antibody. Such binding interactions can be manifested as an intermolecular contact with one or more amino acid residues of one or more CDRs. Antigen binding can involve, for example, a CDR3 or a CDR3 pair or, in some cases, interactions of up to all six CDRs of the V_{H} and V_{L} chains. An epitope can be a linear peptide sequence (i.e., “continuous”) or can be composed of noncontiguous amino acid sequences (i.e., “conformational” or “discontinuous”). An antibody can recognize one or more amino acid sequences; therefore an epitope can define more than one distinct amino acid sequence. Epitopes recognized by antibodies can be determined by peptide mapping and sequence analysis techniques well known to one of skill in the art. Binding interactions are manifested as intermolecular contacts with one or more amino acid residues of a CDR.

The term “specific” refers to a situation in which an antibody will not show any significant binding to molecules other than the antigen containing the epitope recognized by the antibody. The term is also applicable where for example, an antigen binding domain is specific for a particular epitope which is carried by a number of antigens, in which case the antibody will be able to bind to the various antigens carrying the epitope. The terms “preferentially binds” or “specifically binds” mean that the antibodies bind to an epitope with greater affinity than it binds unrelated amino acid sequences, and, if cross-reactive to other polypeptides containing the epitope, are not toxic at the levels at which they are formulated for administration to human use. In one aspect, such affinity is at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences. The terms “immunoreactive,” “binds,” “preferentially binds” and “specifically binds” are used interchangeably herein. The term “binding” refers to a direct association between two molecules, due to, for example, covalent, electrostatic, hydrophobic, and ionic and/or hydrogen-bond interactions under physiological conditions, and includes interactions such as salt bridges and water bridges, as well as any other conventional means of binding.

“Isolated” (used interchangeably with “substantially pure”) when applied to polypeptides means a polypeptide or a portion thereof which, by virtue of its origin or manipulation: (i) is present in a host cell as the expression product of a portion of an expression vector; or (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins. The term generally means a polypeptide that has been separated from other proteins and nucleic acids with which it naturally occurs. Typically, the polypeptide is also separated from substances such as antibodies or gel matrices (polyacrylamide) which are used to purify it.

Also provided herein is an ex vivo method for generating a population of regulatory T lymphocytes comprising culturing T cells with a composition comprising antibodies that specifically bind to CD80 and CD86 in the presence of cells that present either alloantigen or a non-cellular protein antigen. In one embodiment, the non-cellular protein antigen is human gamma globulin, equine gamma globulin or ovalbumin.

The cells may be obtained by the subject to receive an organ or graft transplant. For example, T cells may be obtained from a recipient animal and the cells that present alloantigen are either cells taken from a donor subject or cells pulsed with antigen taken from a donor subject.

Also provided herein is a cell culture prepared by the method described above, comprising culturing cells obtained by the ex vivo method in medium. In one embodiment, the antibodies that specifically bind to CD80 and CD86 are removed from said medium. Removal of antibodies may be by any conventionally accepted laboratory method such as those described in the examples. Antibodies may be removed from the culture medium by, for example, by washing the cells and reconstituting the cells in medium. The cells obtained from these methods may be further administered to a recipient subject in need thereof. Any commercially acceptable medium (e.g., DMEM, RPMI, etc.) may be used to culture the cells under conditions according to conventional laboratory methods.

After culture, T regulatory cells to be used in the treatment methods described herein exhibit cell surface marks such as, for example, CD4⁺, CD25⁺, and Foxp3⁺.

Also provided herein is a method of suppressing rejection of an organ or tissue transplant in a recipient subject comprising the following steps: (a) obtaining a sample of T cells from the recipient subject; (b) obtaining a sample of alloantigen from a donor subject, said donor subject being the source of the organ or tissue being transplanted; (c) exposing said sample of T cells to said sample of alloantigen in the presence of a composition comprising antibodies that specifically bind to CD80 and CD86 to generate a composition comprising a population of regulatory T lymphocytes; and (d) administering to the recipient subject a composition comprising said population of regulatory T-lymphocytes. Step (c) may further comprise removing the antibodies from said composition prior to step (d).

In one embodiment, from about 1×10⁹ to about 1×10¹⁵ cells may be administered to said recipient subject. The number of cells to be administered to a subject may be empirically determined by a medical practitioner based upon the age, weight, height, and general health of the recipient subject. The population of regulatory T-lymphocytes may be administered to the recipient subject prior to, concurrently with, or after, transplant of an organ or tissue (graft). In one embodiment, the subject is a human. Administration of cells or antibodies to a subject may be by any means such as, for example, injection or infusion.

Provided herein are methods for inhibiting or delaying graft or organ rejection to prevent or inhibit various modes of attack, for example, inhibition of T-cell attack, inhibition of antibody responses, and inhibition of cytokine and complement effects. Prescreening of donors to match them with recipients is conducted to help prevent rejection, especially in preventing hyperacute rejection.

For the purposes of the present embodiments, “a therapeutically effective amount” means in the conventional sense, i.e., as an amount sufficient to provide a health benefit to the subject being treated such that, in one embodiment, the transplanted organ is not rejected. In another embodiment, a therapeutically effective amount may be an amount sufficient to provide a health benefit to the subject being treated such that, in one embodiment, the time until rejection is delayed by about 1 month, about 6 months, about 12 months, about 1.5 years, about 2 years, about 3 years, about 4 years, about 5 years, about 10 years, about 15 years, about 20 years or more compared to a subject not receiving treatment.

A recipient described herein may be a recipient of, for example, a hematopoietic cell or bone marrow transplant, an allogeneic transplant of pancreatic islet cells, or a solid organ transplant selected from the group consisting of a heart transplant, a kidney-pancreas transplant, a kidney transplant, a liver transplant, a lung transplant, and a pancreas transplant. Additional examples of grafts or transplants include, but are not limited to, allotransplanted cells, tissues, or organs such as vascular tissue, eye, cornea, lens, skin, bone marrow, muscle, connective tissue, gastrointestinal tissue, nervous tissue, bone, stem cells, cartilage, hepatocytes, or hematopoietic cells. In some embodiments, the graft rejection is an acute humoral rejection of a grafted cell, tissue, or organ. In other embodiments, the graft rejection is a chronic humoral rejection of a grafted cell, tissue, or organ.

In some embodiments, a population of cells described herein are administered prior to a transplant. In other embodiments, the population of cells described herein are administered at the time of transplantation. In other embodiments, the population of cells described herein are administered post-transplant.

Non-limiting examples of specific protocols that may be used in the methods described herein are provided in more detail in the Examples and the figures.

Additional drugs may be utilized, as needed in some instances, to delay graft rejection to prolong graft survival or survival of a recipient of an organ transplant). Any of the methods described herein may be administered in conjunction with another treatment. For example, the patient may be administered one or more immunosuppressive drugs during treatment. An immunosuppressive drug may be one that helps prevent the immune system from rejecting the organ transplant. Non-limiting examples of immunosuppressive drugs include, but are not limited to, a calcineurin inhibitor (e.g., tacrolimus (FK-506), cyclosporine A (CsA), etc.), adriamycin, azathiopurine (AZ), busulfan, cyclophosphamide, deoxyspergualin (DSG); FTY720 (also called Fingolimod, chemical name: 2-amino-2-[2-(4-octylphenyl)ethyl]-1,3-propanediol hydrochloride), fludarabine, 5-fluorouracil, leflunomide (LEF); methotrexate, mizoribine (MZ); mycophenolate mofetil (MMF), a nonsteroidal anti-inflammatory, sirolimus (rapamycin), an adrenocortical steroid (e.g., prednisolone and methylprednisolone), agents that block CTLA-4 and/or CD28, an antibody (e.g., muromonab-CD3, alemtuzumab, basiliximab, daclizumab, rituximab, anti-thymocyte globulin, etc), or a combination thereof.

Cyclosporine A is one of the most widely used immunosuppressive drugs for inhibiting graft rejection by inhibiting interleukin-2 (IL-2) (it prevents mRNA transcription of interleukin-2). More directly, cyclosporine inhibits calcineurin activation that normally occurs upon T cell receptor stimulation. Calcineurin dephosphorylates NFAT (nuclear factor of activated T cells), thereby enabling NFAT to enter the nucleus and bind to interleukin-2 promoter. By blocking this process, cyclosporine A inhibits the activation of the CD4+ T cells and the resulting cascade of events which would otherwise occur. Tacrolimus is another immunosuppressant that acts by inhibiting the production of interleukin-2 via calcineurin inhibition. Rapamycin (Sirolimus), SDZ RAD, and interleukin-2 receptor blockers are drugs that inhibit the action of interleukin-2 and therefore prevent the cascade of events described above. Inhibitors of purine or pyrimidine biosynthesis are also used to inhibit graft rejection. These inhibitors prevent DNA synthesis and thereby inhibit cell division including T cell proliferation. The result is the inhibition of T cell activity by preventing the formation of new T cells. Inhibitors of purine synthesis include azathioprine, methotrexate, mycophenolate mofetil (MMF) and mizoribine (bredinin). Inhibitors of pyrimidine synthesis include brequinar sodium and leflunomide. Cyclophosphamide is an inhibitor of both purine and pyrimidine synthesis. Another method for inhibiting T cell activation is to treat a recipient with antibodies that specifically bind to T cells; for example, OKT3 is a murine monoclonal antibody against CD3. This antibody initially activates T cells through the T cell receptor, and then induces apoptosis of the activated T cell.

Other drugs and methods for delaying allotransplant rejection are available. One approach is to deplete T cells, e.g., by irradiation. Depletion of T cells has often been used in bone marrow transplants, especially if there is a partial mismatch of major HLA. A recipient may be administered an inhibitor (blocker) of the CD40 ligand-CD40 interaction.

In some embodiments, a population of cells described herein and an immunosuppressive agent are administered prior to a transplant. In other embodiments, the population of cells described herein and an immunosuppressive agent are administered at the time of transplantation. In other embodiments, the population of cells described herein and an immunosuppressive agent are administered post-transplant.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Preparation of Regulatory T Cells

Composition

The donor's lymphocytes and patient's lymphocytes are obtained from suspending the cells harvested by culturing for two weeks in the presence of anti-CD80 antibody (2D10.4) and anti-CD86 (IT2.1), in 100 ml saline.

Raw Materials

The main raw materials are as follows.

1. Donor's lymphocytes: peripheral blood mononuclear cells collected in the component blood collection device (more than 4×10⁹),

2. Patient's (recipient) lymphocytes: peripheral blood mononuclear cells collected in the component blood collection device (more than 5×10⁹).

If the amount of lymphocytes is not adequate, the patient's spleen-derived lymphocytes will be added

3. Anti-CD80 antibody (2D10.4) from Bay Bioscience Co., Ltd.; Quality standards: non-GMP manufacturing, endotoxin-free.

4. Anti-CD86 antibody (IT2.2) from Bay Bioscience Co., Ltd.; Quality standards: non-GMP manufacturing, endotoxin-free.

Mechanism of Action

Induction of immune tolerance by the cultured regulatory T cells is expected.

Efficacy

According to mechanism of action described in section 2.4, it is expected to reduce the dose of immunosuppressive agents and to discontinue early after liver transplantation.

Manufacturing Method

The manufacturing method is described in the standard procedure of test article. The main manufacturing processes are as follows.

1. More than 4×10⁹mononuclear cells are collected from donor's peripheral blood using a component blood collection device and are cryopreserved.

2. More than 5×10⁹mononuclear cells are collected from patient's peripheral blood using a component blood collection device. If the count of cells is not enough, you can collect and add the lymphocytes from the spleen removed at the time of liver transplantation.

3. The donor's mononuclear cells 2×10⁹ are thawed, and are co-cultured with the patient's mononuclear cells in the presence of patient plasma, anti-CD86 antibody and anti-CD80 antibody.

4. The cultured cells are recovered after 1 week, and again, the donor's mononuclear cells 2×10⁹ are thawed, and are co-cultured with the patient's mononuclear cells in the presence of patient plasma, anti-CD86 antibody and anti-CD80 antibody.

5. After 1 week, the cultured cells are recovered and washed, and suspended in 100 ml saline.

Quality Standards

The quality standards are described in the standard procedure of test article. The quality standards of the intermediate test article and the final test article are as follows: the samples from the intermediate test article are collected from the culture medium 4 days prior to preparation of the final test article. The samples from the final test article are collected from the cell suspension immediately harvesting the final test article.

| Subject | | | |
|---|---|---|---|
| article | Test items | Test method | Judgment criteria |
| Intermediate | Sterile test | Common bacterial and | No bacteria are |
| test article | | fungal culture | detected |
| | Endotoxin test | Turbidimetric analysis | Below the |
| | | | sensitivity |
| | Mycoplasma | Culture method | Not detected |
| Final test | Cell count test | Blood cell counting | More than |
| article | | device | 1 × 10⁸ |
| | Bacterial test | Common bacterial and | No bacteria are |
| | | fungal culture | detected |
| | Endotoxin test | Colorimetric method | Below the |
| | | | sensitivity |
| | Mycoplasma | Culture method | Not detected |

### Results of Non-clinical Studies

Small-size cultivation test of peripheral blood samples from healthy humans

Test Methods

The peripheral blood is collected from two healthy adults, with one resembling the donor and the other resembling the patient (recipient), and a small number of cells are used. The induction experiment of regulatory T cells is performed with the manufacturing method and the used antibodies as the same conditions. As for the cells before and after culture, we analyzed the cell count and surface antigen, and examined the effect of immunosuppression by MLR.

Induction Results of Regulatory T Cells

The experiment was performed 4 times in accordance with the method described above. The summary of results is shown in Table 1.

Total lymphocyte count reaches 6.80±7.46×10⁶ after two-week culture from 23.89±11.39×10⁶ before culture. This reduction in the number of cells is considered as because of the specific fraction of lymphocytes that cannot survive in the present culture system and the death of other leukocytes.

According to the analysis of the surface antigen, the phenotype of these lymphocytes is analyzed; and according to the 2-week culture of CD3⁺ CD4⁺ cells, an increase of about 15% from 40.83 ±3.52% to 55.01 ±5.39% is recognized. On the other hand, the regulatory T cells, CD4⁺ CD25⁺ Foxp3⁺ cells have a more than 10-fold increase in the ratio from 0.21±0.04% to 2.73±1.27%. Thus, the regulatory T cells are considered as selectively induced by the present culture system. Furthermore, as for the ratio among CD4⁺ cells, it was increased to 6.16±2.01% from 1.29±0.60% for CD4⁺ CD25⁺ Foxp3⁺ cells, to 4.68±1.49% from 1.90±1.27% for CD4⁺ CD25⁺ CTLA4⁺ cells, and to 2.40±2.24% from 1.04±0.79% for CD4⁺ CD127¹° Foxp3⁺ cells respectively.

TABLE 1
Regulatory T cells in cultured cells

| | | At time of | After 2-week |
|---|---|---|---|
| | N | starting culture | culture |
| Lymphocyte count (×10⁶) | 4 | 23.89 ± 11.39 | 6.80 ± 7.46 |
| CD3⁺ CD4⁺ (%) | 4 | 40.83 ± 3.52 | 55.0 ± 5.39 |
| CD4⁺ CD25⁺ Foxp3⁺ (%) | 4 | 0.21 ± 0.04 | 2.73 ± 1.27 |
| CD4⁺ CD25⁺ Foxp3⁺/CD4⁺ (%) | 4 | 1.29 ± 0.60 | 6.16 ± 2.01 |
| CD4⁺ CD25⁺ CTLA4⁺/CD4⁺ (%) | 4 | 1.90 ± 1.27 | 4.68 ± 1.49 |
| CD4⁺ CD127^{lo} Foxp3⁺/CD4⁺ (%) | 4 | 1.04 ± 0.79 | 2.40 ± 2.24 |

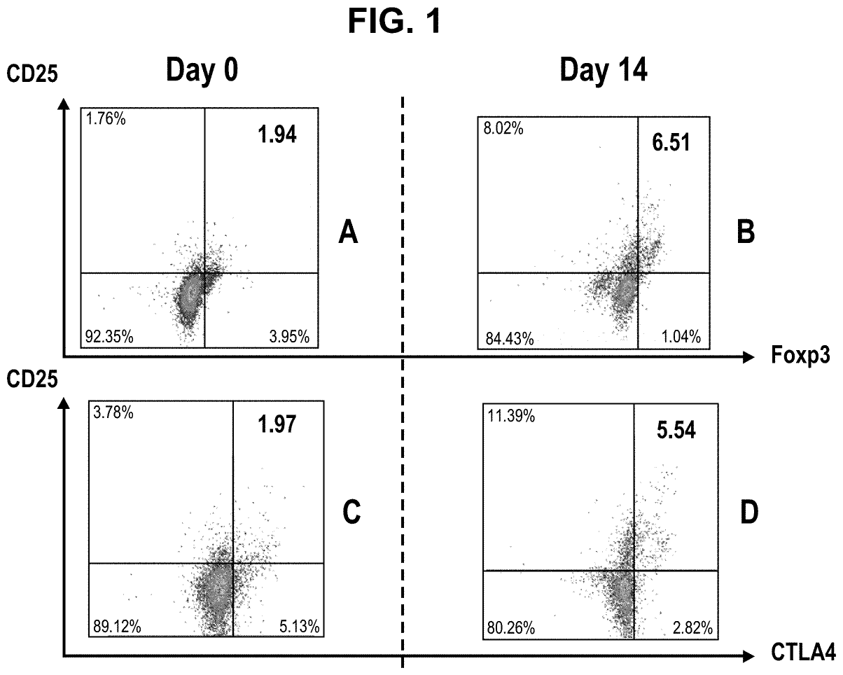

FIG. 1 shows the results of flow cytometry regarding surface antigen analysis before and after a typical culture. This figure shows the CD25⁺ Foxp3⁺ cells and CD25⁺ CTLA4⁺ cells out of CD4⁺ cells.

Properties of Cultured Cells

After culture, the ratio of the regulatory T cells, CD4+ CD25+ Foxp3⁺ cells has increased markedly, but its frequency remains at that few percent. To understand the properties of the test article, we examined the fraction of cells included in the test article by analyzing the surface antigen according to the cultured cells. The examination was performed four times using the cells harvested from the experiment above. The results are shown in Table 2.

Among the cultured cells, the CD4^{+T} cells account for 55.01±5.39%, CD8 T cells account for 26.5±4.68%, and T cells account for more than 80%. In contrast to this, B and NK cells account for 5.98 ±0.85% and 2.81±1.45%, respectively. In addition, monocytes account for 4.83±3.41%. Furthermore, dendritic cells account for 2.3% and granulocytes account for around 0.2%.

TABLE 2
Phenotype of lymphocytes cultured for weeks

| | | | After 2-week |
|---|---|---|---|
| Phenotype | Cell type | N | culture (%) |
| CD3⁺ CD4⁺ | CD4⁺ T cell | 4 | |
| CD3⁺ CD8⁺ | CD8⁺ T cell | 4 | 26.52 ± 4.68 |
| CD3⁻ CD19+ | B cell | 4 | 5.98 ± 0.85 |
| CD3⁻ CD16+ 56⁺ CD45⁺ | NK cell | 4 | 2.81 ± 1.45 |
| CD14⁺ SCC^{mid} | Monocyte | 4 | 4.83 ± 3.41 |
| Lin1⁻ CD11c⁺ HLA-DR⁺ | Myeloid DC | 4 | 1.00 ± 1.65 |
| Lin1⁻ CD123⁺ HLA-DR⁺ | Plasmacytoid DC | 4 | 1.29 ± 1.95 |
| Lin1⁻ CD123⁺ HLA-DR⁻ | Granulocyte | 4 | 0.16 ± 0.09 |

Immunosuppressive Effect of Cultured Cells

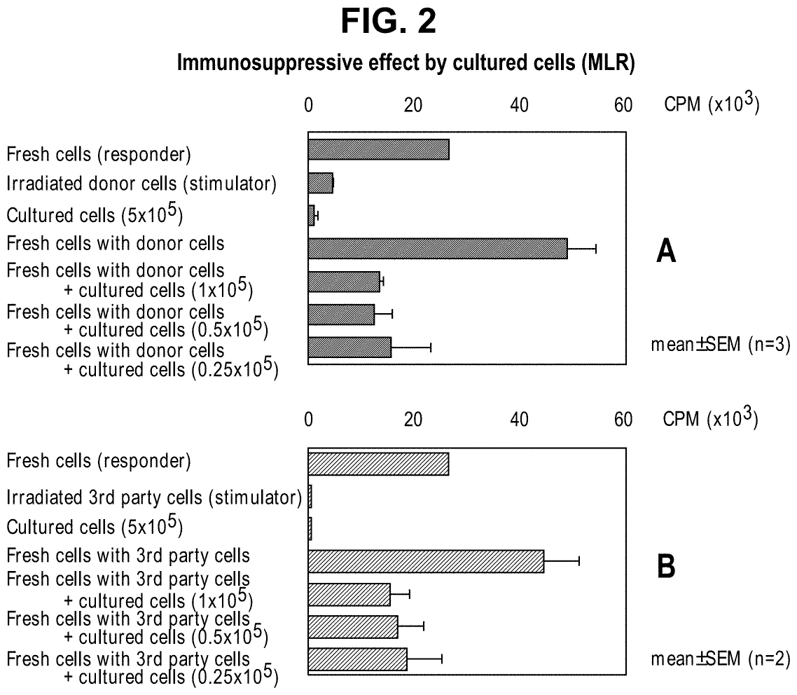

Next, we performed the examination using MLR method to show the immunosuppressive effects of cells containing the induced regulatory T cells. The summary of the results from the three examinations are shown in FIG. 2.

The upper graph of the figure shows the MLR result using the donor's antigen (radiated lymphocytes) used in culture of regulatory T cells, and the lower graph shows the MLR result of antigen from a third party's donor (radiated lymphocytes). The columns 1 to 3 on each graph show the cell proliferation when the recipient's lymphocytes, radiated lymphocytes and 2-week cultured lymphocytes are individually cultured. Column 4 is the cell proliferation of recipient's lymphocyte upon addition of co-culturing and simulating with donor's antigen (radiated lymphocytes) (control). The results of adding 1/1, ½ and ¼ amount of cultured cells in this system are shown in columns 5 to 7, but by adding the cultured cells, the proliferation of lymphocytes is strongly inhibited, and addition of cell count reveals a strong immunosuppressive effect even in ¼ (0.25×10⁵).

Study on Residual Antibodies After Washing

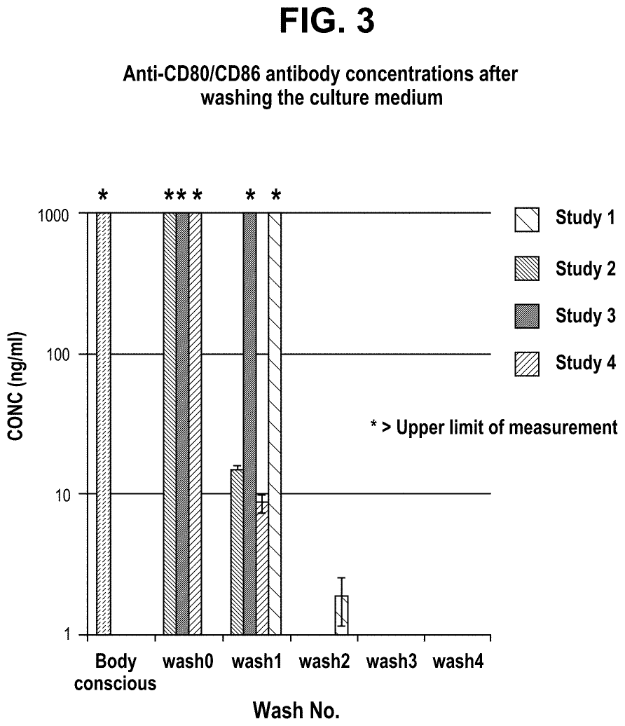

It is not preferable that the antibodies used for culture are left in the final test article to be administered to a patient. For this reason, as for the manufacturing processes of test article in this study, in order to study the number of washes, the number of washes and the residual amounts of the antibody were examined in small trials (n=4). As for the anti-human CD80 and CD86 antibodies used in this study, the isotype is mouse IgG; as for the residual amounts of these antibodies were studied by measuring the mouse IgG using ELISA. Washing was performed for a total of four times, and the residual antibody concentration of each time was studied in the four tests. The results are shown in FIG. 3. The residual antibodies are found in all cases after washing once, the antibodies are not detectable in three 3 out of 4 cases after washing twice, and no residual antibodies are found in any cases after washing more than three times. Therefore, by washing four times in the manufacturing process of test article in this study, it can avoid the risk of antibodies being left in test article and entering to patient's body.

### Large-Size Culture Test of Cells Collected from Healthy Humans by Apheresis

Test Methods

The peripheral monocytes are collected from two healthy adults using the component collection method (apheresis), with one resembling the donor and the other resembling the patient (recipient), and a great number of cells similar like those for an actual cell therapy are used. The induction experiment of regulatory T cells is performed with the manufacturing method and the used antibodies as the same conditions. In addition, as for the cells before and after culture, we analyzed the cell count and surface antigen.

Induction Results of Regulatory T Cells

The experiment was performed in accordance with the method described above. The summary of results is shown in Table 3.

Total lymphocyte count reaches 3.14×10⁹ after two-week culture from 10.95×10⁹ before culture. This reduction in the number of cells is considered as because of the specific fraction of lymphocytes that cannot survive in the present culture system and the death of other leukocytes, which are similar with those in small-sized culture tests. In addition, this cell count is shown in the “4 previously performed clinical studies”. In the regulatory T cell therapy to kidney transplantation of Tokyo Women's Medical College, it is comparable to the number of cells in a real treatment, which is considered to be one of the evidences to properly conduct this test.

According to the analysis of the surface antigen, the phenotype of these lymphocytes is analyzed; and according to the 2-week culture of CD3 CD4 + cells, an increase of about 11% from 43.3% to 54.4% is found. On the other hand, the regulatory T cells, CD4+ CD25+ Foxp3⁺ cells have a more than 12-fold increase in the ratio from 0.36% to 4.41%. Thus, the regulatory T cells are considered as selectively induced by the present culture system, which is similar to the results of the small-size tests.

Furthermore, as for the ratio among CD4⁺ cells, it was increased to 9.2% from 1.2% for CD4⁺ CD25⁺ Foxp3⁺ cells, to 14.1% from 0.7% for CD4⁺ CD25⁺ CTLA4⁺ cells, and to 3.3% from 1.6% for CD4⁺ CD127¹° Foxp3⁺ cells respectively (Table 3).

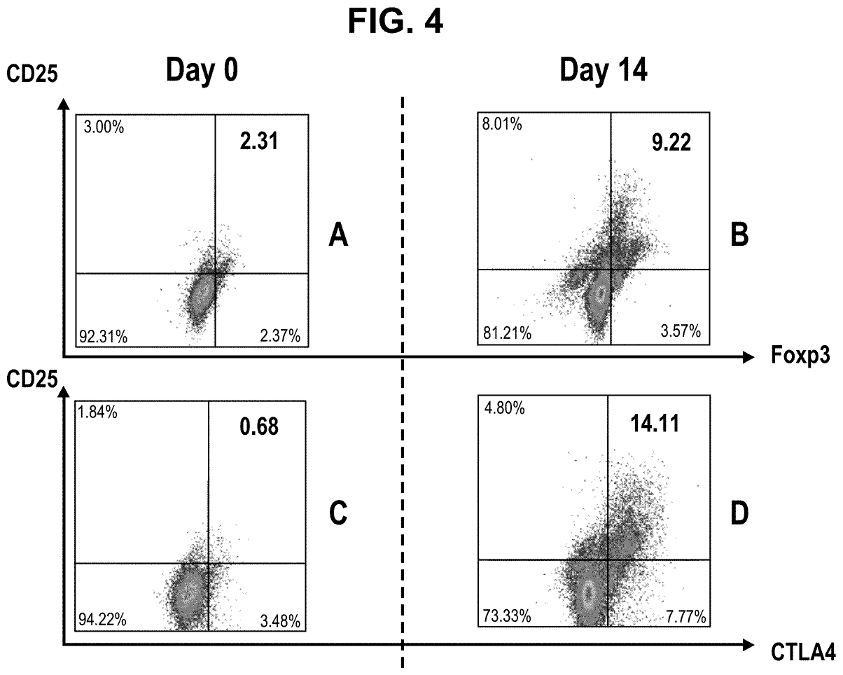

FIG. 4 shows the results of flow cytometry regarding surface antigen analysis before and after a typical culture. This figure shows the CD25⁺ Foxp3⁺ cells and CD25+ CTLA4⁺ cells out of CD4⁺ cells.

These results are shown in the “4 previously performed clinical studies”. In the regulatory T cell therapy to kidney transplantation of Tokyo Women's Medical College, the cells are almost identical to those in the actual treatment. It is expected to obtained the equivalent efficacy to the clinical studies previously performed at Tokyo Women's Medical College using the cells obtained from this study.

TABLE 3
Regulatory T cells in cultured cells

| | At time of | After 2-week |
|---|---|---|
| | starting culture | culture |
| Lymphocyte count (×10⁹) | 10.95 | 3.14 |
| CD3⁺ CD4⁺ (%) | 43.32 | 54.42 |
| CD4⁺ CD25⁺ Foxp3⁺ (%) | 0.36 | 4.41 |
| CD4⁺ CD25⁺ Foxp3⁺/CD4⁺ (%) | 1.20 | 9.22 |
| CD4⁺ CD25⁺ CTLA4⁺/CD4⁺ (%) | 0.68 | 14.11 |
| CD4⁺ CD127^{lo} Foxp3⁺/CD4⁺ (%) | 1.63 | 3.25 |

Properties of Cultured Cells

After culture, the ratio of the regulatory T cells, CD4⁺ CD25⁺ Foxp3⁺ cells has increased markedly, but its frequency remains at that few percent. To understand the properties of the test article, we examined the fraction of cells included in the test article by analyzing the surface antigen according to the cultured cells. The results are shown in Table 4.

Among the cultured cells, the CD4 T cells account for 54.4%, CD8⁺ T cells account for 30.0%, and T cells account for more than 84%. In contrast to this, B and NK cells account for 6.5% and 7.4%, respectively. In addition, monocytes account for 1%. Furthermore, dendritic cells account for 0.2% and granulocytes account for around 0.1%. These results are almost same as those in small tests.

TABLE 4
Phenotype of cultured lymphocytes

| | | After 2-week |
|---|---|---|
| Phenotype | Cell type | culture |
| CD3⁺ CD4⁺ | CD4⁺ T cell | 54.42 |
| CD3⁺ CD8⁺ | CD8⁺ T cell | 30.01 |
| CD3⁻ CD19⁺ | BceU | 6.52 |
| CD3⁻ CD16⁺ 56⁺ CD45⁺ | NK cell | 7.42 |
| CD14⁺ SCC^{mid} | Monocyte | 1.02 |
| Lin1⁻ CD11c⁺ HLA-DR⁺ | Myeloid DC | 0.12 |
| Lin1⁻ CD123⁺ HLA-DR⁺ | Plasmacytoid DC | 0.07 |
| Lin1⁻ CD123⁺ HLA-DR⁻ | Granulocyte | 0.13 |

### T-Regulatory Cell Therapy Study Using Animals

Studies Using Mouse

The present inventors have succeeded in inducing the antigen specific Treg-like cells ex vivo by adding anti-CD80 and anti-CD86 antibodies in lymphocyte culture medium, and have confirmed the long-term graft survival in mouse heart transplantation model by infusing the harvested cells (Ref 6).

Studies Using Monkey

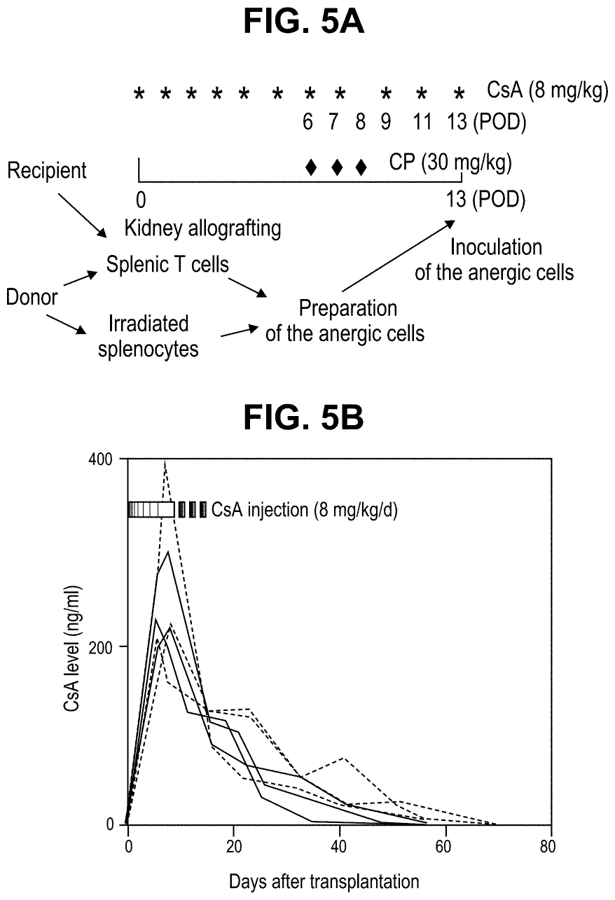

In a study trying the same protocol for non-clinical studies using the monkey kidney transplantation model, by infusion of regulatory T cells harvested from co-culturing and inducing peripheral blood mononuclear cells (PBMC) under the donor's splenocytes and anti-CD80/CD86 antibodies, into the recipient before and after 2 weeks after transplantation, the early withdrawal of immunosuppressive agent, cyclosporine has been enabled at about day 60 after the procedure, and then the transplanted kidney has achieved long-term engraftment even in a immunosuppression free state (FIG. 5) and in the following table which provides the data for the treatment and outcome of transplanted monkeys.

| | Origin of | | | | | |
|---|---|---|---|---|---|---|
| | the stimulators | mAbs added | | Lymphocyte | Number of | |
| | in | in the culture | Administration | cell | inoculated | Survival |
| Group | culture | medium | of CP | count (/mm³) | cells (×10⁶) | (d) |
| A | Donor | anti- | (+) | 662 ± 239 | 102 ± 67 | 75,^{A,B} 81,^{C} 212,^{D} >410, |
| | | CD80/CD86 | | | | >840, >880 |
| | | mAbs | | | | |
| B | (−) | (−) | (+) | 550 ± 192 | 0 | 15,^{A} 16,^{A} 26,^{A} 28,^{A} 28^{A} |
| C | Third-party | anti- | (+) | 718 ± 132 | 79 ± 22 | 28,^{A} 33,^{A} 41,^{A} 60,^{A} 73^{A} |
| | | CD80/CD86 | | | | |
| | | mAbs | | | | |
| D | Donor | anti- | (−) | 3920 ± 120 | 90 ± 5 | 27,^{A} 28^{A} |
| | | CD80/CD86 | | | | |
| | | mAbs | | | | |
| E | Donor | anti-CD86 | (+) | 465 ± 150 | 155 ± 55 | 43,^{A} 67,^{A} 69,^{A} 111^{A} |
| | | mAbs | | | | |
| F | Donor | anti-CD80 | (+) | 300 | 150 | 43^{A} |
| | | mAbs | | | | |

Values are expressed as mean ± SD.
All recipients were splenectomized.
Numbers in Survival column represent values for each animal in the group.
^{A}Died of acute rejection;
^{B}received an inoculation of 4 × 10⁶ cells;
^{C}died of bleeding after renal biopsy;
^{D}died of hydronephrosis due to urethral stenosis.

After the donor's and third parity's skin grafting are performed in these animals, the donor's skin arrives, but the skin of third party is rejected, which showing the donor's antigen specific immune tolerance induction (Ref 7).

### Results of Previous Clinical Studies

Regulatory T Cell Therapy in Kidney Transplantation (Phase I Study)

Based on the results of pre-clinical studies of cell therapy using the induced regulatory T cells, the co-investigators, Teraoka et al. from Kidney Center of Tokyo Women's Medical College tried the same treatment for 9 cases of living-donor kidney transplantation from August 2008 to October 2009 (Ref 8).

Patient Background

The background of patients tested in the phase I clinical study is shown in Table 5. The patients are aged between 26 and 53, and other information such as underlying disease is shown in Table 5.

TABLE 5
Patient background

| | Age | Transplantation | Underlying | History of | | Blood | HLA |
|---|---|---|---|---|---|---|---|
| | Gender | date | disease | dialysis | Donor | group | mm |
| 1 | 42 | Aug. 28, 2008 | IgA | Not | Yong | A→A | 3 |
| | M | | nephropathy | introduced | sister | | |
| 2 | 46 | Feb. 5, 2009 | Mitochondria | 2 y 3 m | Husband | O→B | 6 |
| | F | | genetic | | | | |
| | | | abnormality | | | | |
| | | | DM | | | | |
| 3 | 53 | Apr. 9, 2009 | CGN | 1 y 2 m | Elder | O→AB | 1 |
| | M | | | | sister | | |
| 4 | 41 | May 21, 2009 | IgA | 2 y 7 m | Mother | AB→AB | 2 |
| | F | | nephropathy | | | | |
| 5 | 26 | Jun. 18, 2009 | IgA | 2 y 5 m | Mother | A→A | 3 |
| | M | | nephropathy | | | | |
| 6 | 36 | Jul. 9, 2009 | CGN | 7 y 10 m | Mother | B→AB | 3 |
| | M | | | | | | |
| 7 | 47 | Aug. 13, 2009 | IgA | 11 m | Yong | O→O | 3 |
| | F | | nephropathy | | sister | | |
| 8 | 53 | Sep. 3, 2009 | IgA | 17 y 4 m | Wife | A→A | 3 |
| | M | | nephropathy | | | | |
| 9 | 34 | Oct. 1, 2009 | CGN | 7 y 8 m | Father | B→B | 2 |
| | M | | | | | | |

Typical Clinical Course

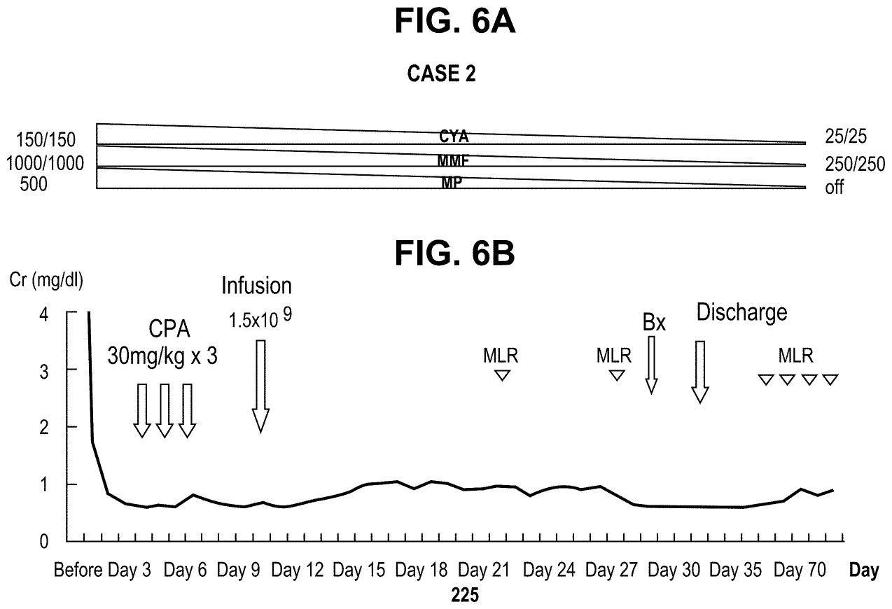

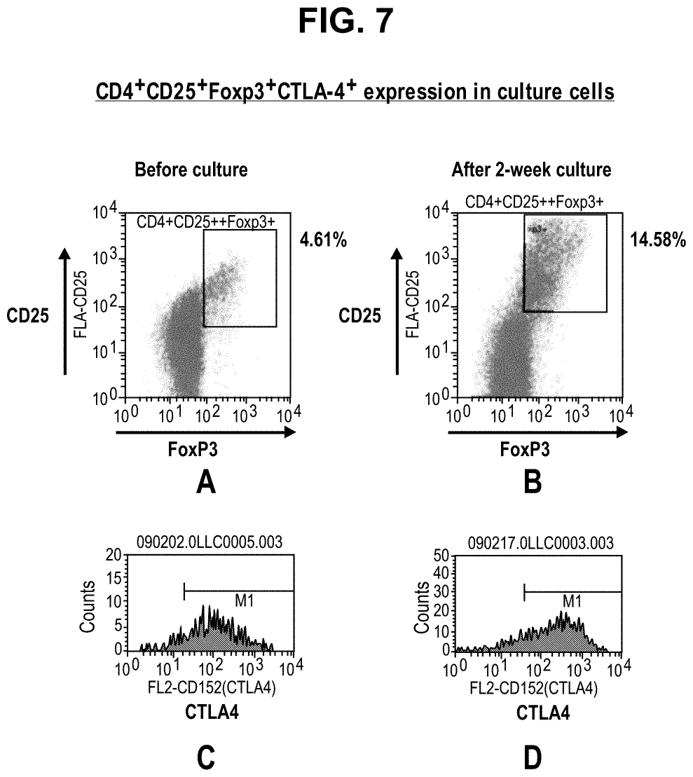

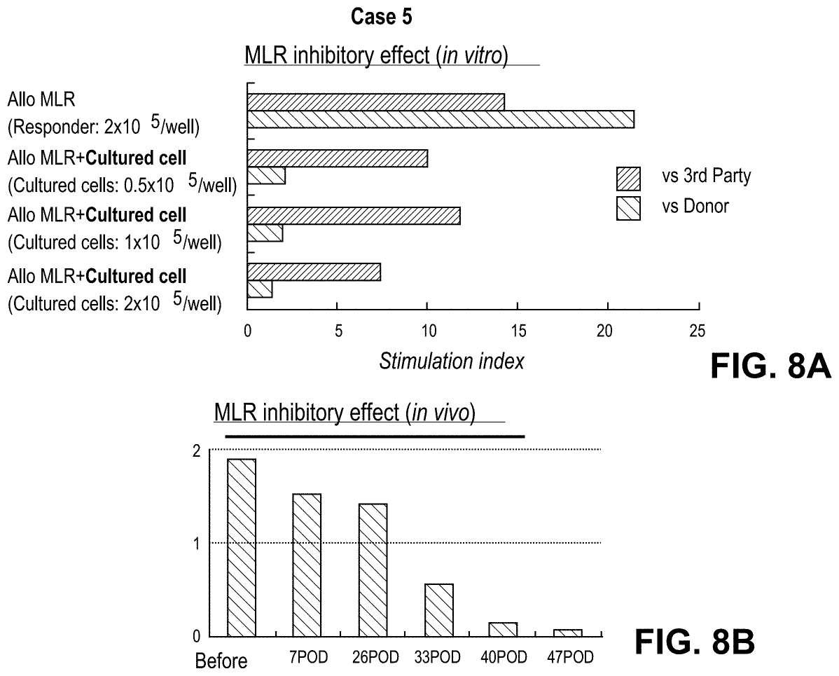

As can be seen in Case 2 (FIG. 6), 1.5×10⁹ regulatory T cells are infused at week 2 after surgery, and the immunosuppressive agent (cyclosporin (CYA): 300 mg/day, mycophenolate mofeteil (MMF): 2000 mg/day, methylprednisolone (MP): 500 mg/day) are reduced gradually. Now, the doses of day 225 are CYA 50mg/day and MMF 500 mg/day, and MP is completely discontinued. During the same period, no acute rejection or apparent side effects have been observed in renal function and renal biopsy. Doses of immunosuppressive drugs have also been successfully reduced by ½ to ⅕ in other cases. For these cases, no acute rejection or apparent side effects same as those in case 2 have been observed in renal function and renal biopsy. The results of flow cytometry of induced regulatory T cells are shown in FIG. 7. These cells are in vitro and in kidney transplant patients (case 5), inhibit the donor's antigen-specific lymphocyte proliferation (FIG. 8).

Results of Cell Culture

The results of 9 cases of cell culture performed in the phase I clinical study is shown in Table 6. Total lymphocyte count reaches 1.08±0.51 ×10⁹ after two-week culture from 6.50±1.17 ×10⁹ before culture. This reduction in the number of cells is considered as because of the specific fraction of lymphocytes that cannot survive in the present culture system and the death of other leukocytes, which are similar with the results of non-clinical studies.

According to the analysis of the surface antigen, the phenotype of these lymphocytes is analyzed; and according to the 2-week culture of CD3⁺ CD4⁺ cells, an increase of about 5% from 36.69±9.83% to 41.15±11.18% is recognized. On the other hand, the regulatory T cells, CD4⁺ CD25+ Foxp3⁺ cells have a more than 1.5-fold increase in the ratio from 3.04±1.53% to 1.84±0.57%. Thus, the regulatory T cells are considered as selectively induced by the present culture system, which is similar to the results of non-clinical studies.

Furthermore, as for the ratio among CD4⁺ cells, it was increased to 8.87±3.41 from 4.63±0.03% for CD4⁺ CD25⁺ Foxp3⁺ cells, and to 8.54±3.29% from 4.09±0.13% for CD4⁺ CD25⁺ CTLA4⁺ cells respectively.

TABLE 6
Regulatory T cells in cultured cells (data from the
clinical studies of Tokyo Women's Medical College)

| | | At time of | After 2-week |
|---|---|---|---|
| | N | starting culture | culture |
| Lymphocyte count (×10⁹) | 9 | 6.50 ± 1.17 | 1.08 ± 0.51 |
| CD3⁺ CD4⁺ (%) | 3 | 41.15 ± 11.18 | 36.69 ± 9.83 |
| CD4⁺ CD25⁺ Foxp3⁺ (%) | 3 | 1.84 ± 0.57 | 3.04 ± 1.53 |
| CD4⁺ CD25⁺ Foxp3⁺/CD4⁺ (%) | 3 | 4.63 ± 0.03 | 8.87 ± 3.41 |
| CD4⁺ CD25⁺ Foxp3⁺ CTLA4⁺/ | 3 | 4.09 ± 0.13 | 8.54 ± 3.29 |
| CD4⁺ (%) | | | |

Adverse Events

In the phase I clinical study, adverse events determined to be caused by or to have a casual relationship with the regulatory T cells have not been reported.

References

1. Todo S, Fung J J, Starzl T E, Tzakis A, Doyle H, Abu-Elmagd K et al. Single-center experience with primary orthotopic liver transplantation with FK 506 immunosuppression. Ann Surg 1994; 220 (3): 297-308; discussion 308-299.

2. Furukawa H, Todo S. Evolution of immunosuppression in liver transplantation: contribution of cyclosporine. Transplant Proc 2004; 36 (2 Suppl): 274S-284S.

3. Okumura K, Herzenberg L A, Murphy D B, McDevitt H O. Selective expression of H-2 (i-region) loci controlling determinants on helper and suppressor T lymphocytes. J Exp Med 1976; 144 (3): 685-698.

4. Sakaguchi S, Sakaguchi N, Asano M, Itoh M, Toda M. Immunologic self-tolerance maintained by activated T cells expressing IL-2 receptor alpha-chains (CD25). Breakdown of a single mechanism of self-tolerance causes various autoimmune diseases. J Immunol 1995; 155 (3): 1151-1164.

5. Wood K J, Sakaguchi S. Regulatory T cells in transplantation tolerance. Nat Rev Immunol 2003; 3(3):199-210.

6. Bashuda H, Seino K, Kano M, Sato K, Azuma M, Yagita H et al. Specific acceptance of cardiac allografts after treatment with antibodies to CD80 and CD86 in mice. Transplant Proc 1996; 28 (2): 1039-1041.

7. Bashuda H, Kimikawa M, Seino K, Kato Y, Ono F, Shimizu A et al. Renal allograft rejection is prevented by adoptive transfer of anergic T cells in nonhuman primates. J Clin Invest 2005; 115 (7): 1896-1902.

8. Ichiro Koyama. Introduction of peripheral immune tolerance in SS3-8 kidney transplantation, the 45t^{h} General Meeting of the Japan Society for Transplantation, 2009.

### Ex vivo Tolerance Induction

This experiment discusses the successful reduction and cessation of immunosuppressants by a regulatory T cell-based cell therapy in living donor liver transplantation.

Purpose: Life-long use of immunosuppressants (IS) is associated with significant immunological and non-immunological adverse effects. Thus, minimization, followed by complete cessation of IS has been an ultimate goal in organ transplantation.

Infusion of ex vivo generated donor-antigen-specific regulatory T cells (Tregs) allows for early withdrawal of IS and induction of tolerance after renal transplantation in non-human primates.

The present study was conducted to determine the safety and the efficacy of Treg-based cell therapy in living donor liver transplantation (LDLT).

Methods: The study was performed with 10 consecutive adult LDLTs. On the day before LDLT, Tregs were started to generate ex vivo for 2 weeks by co-culturing recipient-PBMCs (+splenocytes) with irradiated donor-PMBCs, anti-CD80 mAbs and anti-CD86 mAbs. Immunosuppressants were administered immediately after transplantation. Immunosuppressants were steroid+MMF+tacrolimus (TAC) or cyclosporine (CYA), while the former two (steroid+MMF) were stopped within a month. Cyclophosphamide (40 mg/kg) was given on post-operative day (POD) 5, and Tregs were infused on POD 13. TAC (or CYA) was maintained until 6 months, from when it was reduced every 2-3 months as follows; once daily, and then thrice-, twice- and once-weekly, and finally stopped.

Results: Cases 1-9, except for the case 5 maintained good liver function during reduction and after cessation of Treg infusion. Case 5 was replaced on regular IS and excluded from the study due to inappropriate generation of Tregs. No adverse events were observed in all patients.

Two-week co-culture increased CD4⁺CD25⁺Foxp3⁺ (6.7±3.8% to 28.1±7.7%) and CD4+CD127¹°Foxp3⁺ T cells (8.2±6.0% to 26.2±7.7%). Cultured cells inhibited mixed lymphocyte reaction (MLR) in a cell-number-dependent fashion.

| | Age/ | | Infused | Time after | AST/ALT | |
|---|---|---|---|---|---|---|
| Case | Gender | Disease | cells (×10⁹) | LTx (days) | (IU/L) | IS status |
| 1 | 39/M | LC (HCV) | 0.61 | 731 | 26/18 | Off (for 90 |
| | | | | | | days) |
| 2 | 63/M | LC (alcoholic) | 2.54 | 654 | 36/56 | Off (for 75 |
| | | | | | | days) |
| 3 | 56/M | LC (NASH) | 0.79 | 626 | 18/13 | Off (for 64 |
| | | | | | | days) |
| 4 | 59/M | LC (HBV) + | 2.45 | 521 | 16/9 | TAC 3 mg, |
| | | HCC | | | | x1/wk |
| 5 | 52/M | PBC | 0.63 | 437 | 28/30 | TAC 5 mg, |
| | | | | | | qd |
| 6 | 55/F | PSC | 1.18 | 395 | 24/17 | CYA 150 mg, |
| | | | | | | bid |
| 7 | 59/F | KC (NASH) + | 2.59 | 374 | 22/14 | TAC 2 mg, |
| | | HCC | | | | x3/wk |
| 8 | 56/M | LC (alcoholic) | 0.70 | 297 | 18/12 | CYA 100 mg, |
| | | | | | | bid |
| 9 | 58/F | PBC | 1.10 | 234 | 21/14 | TAC 2 mg/ |
| | | | | | | qd |
| 10 | 55/M | KC (NASH) + | 1.20 | 129 | 29/26 | TAC 3 mg, |
| | | HCC | | | | bid |

Conclusion: Cell therapy based on ex vivo generated donor-antigen specific Tregs allowed early reduction of IS in 9 out of 10 cases and ultimate cessation in 3 out of 10 cases after LDLT.

While certain embodiments of the present application have been shown and described herein, it will be obvious that such embodiments are provided by way of example only. Numerous variations, changes, and substitutions may occur to those skilled in the art without departing from the embodiments; it should be understood that various alternatives to the embodiments described herein may be employed in practicing the methods described herein.

1-22. (canceled)

23. A pharmaceutical composition for inducing generation of a population of regulatory T-lymphocytes by ex vivo exposure to a sample of peripheral blood mononuclear cells (PBMCs) from a donor subject, the donor subject being the source of an organ or tissue being transplanted, and a sample of PBMCs from a recipient subject to produce a drug for treating a condition in a subject mediated by an immune response, the pharmaceutical composition comprising an antibody, or an antigen-binding fragment thereof, that specifically binds to CD80 and an antibody, or an antigen-binding fragment thereof, that specifically binds to CD86, or an antibody, or an antigen-binding fragment thereof, that specifically binds to CD80 and CD86, and a plasma of the recipient subject, wherein the population of regulatory T-lymphocytes is produced by exposing the sample of PBMCs from the recipient subject to the pharmaceutical composition for a week ex vivo in the presence of the sample of PBMCs from the donor subject, and subsequently exchanging the sample of PBMCs from the donor subject and the pharmaceutical composition with a newly prepared sample of PBMCs from the donor subject and pharmaceutical composition, and then continuously exposing the sample of PBMCs from the recipient subject for one more week ex vivo, and wherein the population of regulatory T-lymphocytes is administered to the recipient subject at a cell count of from about 1×10⁹ to about 1×10¹⁵.

24. The composition of claim 23, wherein the antibody, or antigen-binding fragment thereof, that specifically binds to CD80 binds to one or more epitopes on CD80, the antibody, or antigen-binding fragment thereof, that specifically binds to CD86 binds to one or more epitopes on CD86, and the antibody, or antigen-binding fragment thereof, that specifically bind to CD80 and CD86 binds to one or more epitopes on CD80 and to one or more epitopes on CD86.

25. The composition of claim 23, wherein the antibody, or antigen-binding fragment thereof, that specifically binds to CD80 blocks and/or neutralizes CD80, the antibody, or antigen-binding fragment thereof, that specifically binds to CD86 blocks and/or neutralizes CD86, and the antibody, or antigen-binding fragment thereof, that specifically bind to CD80 and CD86 blocks and/or neutralizes CD80 and CD86.

26. An ex vivo method for generating a population of regulatory T lymphocytes, comprising culturing peripheral blood mononuclear cells (PBMCs) from a recipient subject, with a composition that comprises an antibody, or an antigen-binding fragment thereof, that specifically binds to CD80 and an antibody, or an antigen-binding fragment thereof, that specifically binds to CD86, or an antibody, or an antigen-binding fragment thereof, that specifically bind to CD80 and CD86, and a plasma of the recipient subject in the presence of peripheral blood mononuclear cells (PBMCs) from a donor subject for a week, and subsequently exchanging the cells from the donor subject and the composition with a newly prepared cells from the donor subject and composition, and then continuously culturing the PBMCs from the recipient subject for one more week, and wherein the population of regulatory T-lymphocytes is administered to the recipient subject at a cell count of from about 1×10⁹ to about 1×10¹⁵.

27. The ex vivo method of claim 26, wherein the regulatory T lymphocytes produced by the method are formulated for administration to a subject in need thereof.

28. A cell culture prepared by the method of claim 26, comprising the population of regulatory T lymphocytes and medium, wherein the antibody, or antigen-binding fragment thereof, is removed from the medium by washing, wherein the cell from the donor subject is a cell irradiated with radiation.

29. A composition for suppressing rejection of an organ or tissue transplant in a recipient subject, comprising a population of regulatory T lymphocytes,
wherein the population of regulatory T-lymphocytes is produced by a method comprising exposing a sample of peripheral blood mononuclear cells (PBMCs) from the recipient subject to a sample of PBMCs from a donor subject, the donor subject being the source of an organ or tissue being transplanted, in the presence of a composition that comprises an antibody, or an antigen-binding fragment thereof, that specifically binds to CD80 and an antibody, or an antigen-binding fragment thereof, that specifically binds to CD86, or an antibody, or an antigen-binding fragment thereof, that specifically binds to CD80 and CD86, and a plasma of the recipient subject ex vivo for a week, and subsequently exchanging the sample of PBMCs from the donor subject and the composition that comprises an antibody or antibody-binding fragment thereof with a newly prepared sample of PBMCs from the donor subject and composition that comprises an antibody or antibody-binding fragment thereof, and then continuously culturing the sample of PBMCs from the recipient subject for one more week,
wherein the population of regulatory T-lymphocytes is administered to the recipient subject at a cell count of from about 1×10⁹ to about 1×10¹⁵,
wherein the method further comprises removing the antibody, or antigen-binding fragment thereof, from the composition, and
wherein the sample of PBMCs from the donor subject is a cell irradiated with radiation.

30. The composition of claim 29, wherein the composition is administered to the recipient subject prior to, concurrently with, or after, transplant of an organ or tissue.

31. The composition of claim 29, further characterized in that one or more immunosuppressive drugs is administered to the recipient subject.

32. The composition of claim 31, wherein the one or more immunosuppressive drugs is a calcineurin inhibitor, adriamycin, azathiopurine (AZ), busulfan, cyclophosphamide, deoxyspergualin (DSG); FTY720 (2-amino-2-[2-(4-octylphenyl)ethyl]-1,3-propanediol hydrochloride), fludarabine, 5-fluorouracil (5-FU), leflunomide (LEF), methotrexate, mizoribine (MZ), mycophenolate mofetil (MMF), a nonsteroidal anti-inflammatory, sirolimus (rapamycin), an adrenocortical steroid, agents that block CTLA-4, agents that block CD28, an antibody, or a combination thereof.

33. The composition of claim 31, wherein the one or more immunosuppressive drugs is administered to the recipient subject prior to, concurrently with, or after, transplant of the organ or tissue.

34. The composition of claim 32, wherein the calcineurin inhibitor is tacrolimus (FK-506) or cyclosporine A (CsA).

35. The composition of any one of claims 29, wherein the recipient subject is characterized in that the transplanted organ or tissue property functions even if the one or more immunosuppressive drugs is discontinued.

36. The composition of any one of claims 29, wherein the composition is administered to the recipient subject 13 days post-transplantation.

37. The composition of any one of claims 29, wherein the composition comprises CD4+CD25+Foxp3+ cells.

38. The composition of claim 35, wherein a period during which the one or more immunosuppressive drugs is discontinued is at most 90 days.
